(19) United States
(12) Patent Application Publication
(10) Pub. No.: US 2026/0263007 A1
(43) Pub. Date: Sep. 10, 2026

(54) FLEXIBLE ELECTRODE FOR SPINAL CORD AND METHOD FOR MANUFACTURING SAME

(71) Applicant: SHANGHAI STAIRMED TECHNOLOGY CO., LTD., Shanghai (CN)

(72) Inventors: Xue LI; Zhengtuo ZHAO, Shanghai (CN); Xiaocheng LI, Shanghai (CN); Jie FAN, Shanghai (CN)

(21) Appl. No.: 18/875,497

(22) PCT Filed: Jun. 29, 2022
(86) PCT No.: PCT/CN2022/102350

§ 371 Date: Dec. 16, 2024

(30) Foreign Application Priority Data
Jun. 17, 2022 (CN) 202210689989.6

(51) Int. Cl. A61B 5/00; A61B 5/251; A61B 5/279; A61N 1/05

(52) U.S. Cl. CPC A61B 5/686; A61B 5/251; A61B 5/279; A61B 5/6877; A61N 1/0551; A61B 2562/046; A61B 2562/125; A61B 2562/164; A61B 2562/227

(57) ABSTRACT

The present disclosure relates to a flexible electrode for spinal cord and a method for manufacturing the same. There is provided a flexible electrode for spinal cord that includes an attachment portion configured to be capable of being outside an epidural or intradural white matter and configured to acquire or apply an electrical signal on a surface of the spinal cord; wherein the attachment portion of the flexible electrode includes first and second insulating layers and a wire layer located therebetween; wherein the attachment portion of the flexible electrode further includes one or more electrode sites, each of which is electrically coupled to one of wires in the wire layer, and is in contact with the spinal cord after implantation of the flexible electrode to acquire or apply an electrical signal from or to a spinal nerve.

### CROSS-REFERENCE TO RELATED APPLICATIONS

This application is based on and claims the priority to the Chinese application No. 202210689989.6 filed on Jun. 17, 2022, the disclosure of which is hereby incorporated herein by reference in its entirety.

### TECHNICAL FIELD

The present disclosure relates to the field of life science technology, and more particularly, to a flexible electrode for spinal cord and a method for manufacturing the same.

### BACKGROUND

Spinal nerve electrodes include epidural electrodes and intraspinal electrodes.

### SUMMARY

A brief overview of the present disclosure is given below in order to provide a basic understanding of some aspects of the present disclosure. However, it should be understood that this overview is not an exhaustive overview of the present disclosure. It is not intended to identify the key or important parts of the present disclosure, nor is it intended to limit the scope of the present disclosure. Its purpose is simply to give some concepts of the present disclosure in a simplified form as a prelude to a more detailed description given later.

According to a first aspect of the present disclosure, there is provided a flexible electrode for spinal cord, which includes an attachment portion configured to be capable of being outside an epidural or intradural white matter and configured to acquire or apply an electrical signal on a surface of the spinal cord; wherein the attachment portion of the flexible electrode includes a first insulating layer, a second insulating layer, and a wire layer located between the first insulating layer and the second insulating layer; and wherein the attachment portion of the flexible electrode further includes one or more electrode sites, each of which is electrically coupled to one of wires in the wire layer and is in contact with the spinal cord after implantation of the flexible electrode to acquire an electrical signal from a spinal nerve and transmit the acquired electrical signal via the wire, or to apply an electrical signal received via the wire to the spinal nerve.

According to a second aspect of the present disclosure, there is provided a method for manufacturing a flexible electrode for spinal cord according to the first aspect of the present disclosure, which includes: forming the first insulating layer, the wire layer, the second insulating layer, and the electrode site over a substrate; and separating the flexible electrode from the substrate, wherein a through hole is formed at a position corresponding to the electrode site in at least one of the first insulating layer or the second insulating layer by patterning.

Other features of the present disclosure and advantages thereof will become more apparent from the following detailed description of exemplary embodiments of the present disclosure with reference to the accompanying drawings.

### BRIEF DESCRIPTION OF THE DRAWINGS

The accompanying drawings, which constitute a part of the specification, illustrate embodiments of the present disclosure and, together with the description, serve to explain the principles of the present disclosure.

The present disclosure can be more clearly understood from the following detailed description with reference to the accompanying drawings, in which:

FIG. 1 shows a schematic diagram of at least a part of a flexible electrode for spinal cord according to an embodiment of the present disclosure;

FIG. 2 shows a schematic diagram of an implantation method of at least a part of a flexible electrode for spinal cord according to an embodiment of the present disclosure;

FIG. 3 shows an exploded view of at least a part of a flexible electrode according to an embodiment of the present disclosure;

FIG. 4 shows a flowchart of a method for manufacturing a flexible electrode according to an embodiment of the present disclosure;

FIG. 5 shows a schematic diagram of a method for manufacturing a flexible electrode according to an embodiment of the present disclosure;

FIG. 6 shows a schematic diagram of a method for manufacturing a flexible electrode according to an embodiment of the present disclosure;

FIG. 7 shows a schematic diagram of a method for manufacturing a flexible electrode according to an embodiment of the present disclosure.

### DETAILED DESCRIPTION

The following detailed description is made with reference to the accompanying drawings, and is provided to assist in a comprehensive understanding of various exemplary embodiments of the present disclosure. The following description includes various details to assist in understanding, but these details are considered to be examples only and not to limit the present disclosure, and the present disclosure is defined by the appended claims and their equivalents. The words and phrases used in the following description are only used to enable a clear and consistent understanding of the present disclosure. In addition, for clarity and brevity, descriptions of well-known structures, functions, and configurations may be omitted. Those of ordinary skill in the art will recognize that various changes and modifications may be made to the examples described herein without departing from the spirit and scope of the present disclosure.

The following description of at least one exemplary embodiment is in fact merely illustrative and is in no way intended to limit the present disclosure and its application or use. That is, structures and methods herein are shown in an exemplary manner to illustrate different embodiments of the structures and methods in the present disclosure. However, those skilled in the art will appreciate that they merely illustrate exemplary ways of the present disclosure that can be implemented, rather than exhaustive ways. In addition, the drawings need not be drawn to scale, and some features may be enlarged to illustrate the details of specific components.

Technologies, methods, and devices known to those of ordinary skills in the relevant field may not be discussed in detail, but where appropriate, such technologies, methods, and devices should be considered as a part of the granted specification.

In all examples shown and discussed herein, any specific values should be interpreted as merely exemplary and not as limiting. Therefore, other examples of exemplary embodiments may have different values.

Compared with intraspinal electrodes, epidural electrodes can only record local field potential (LFP) signals due to their poor spatial resolution, while the intraspinal electrodes can simultaneously record LFP signals and single neuron Spike signals with higher spatial precision. However, current intraspinal electrodes also face various challenges. Specifically, electrodes with a small number of channels are limited by a number of recorded neuronal activities and cannot fully utilize their spatial resolution for motor decoding and fine limb manipulation; in addition to the channel count, when the electrode is implanted into spinal cord that has been repeatedly deformed, the physical brittleness caused by the stiffness of the electrode material may cause the electrode to break at the electrical interface and during real-time decoding, resulting in the inability of stable and long-term recording; and for stimulation, the stimulation accuracy is low, and only nerve bundles can be stimulated, which are far away from the motor nerves.

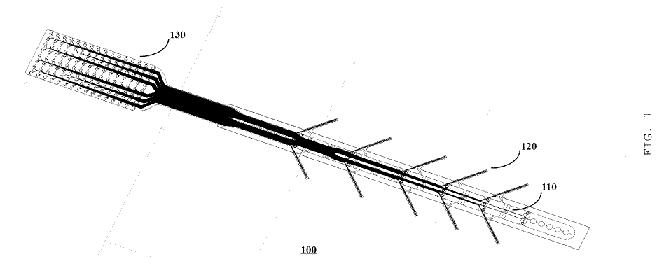

FIG. 1 shows a schematic diagram of at least a part of a flexible electrode 100 for spinal cord according to an embodiment of the present disclosure. As shown in FIG. 1, the flexible electrode 100 may include an attachment portion 110, which may be configured to be capable of being located outside an epidural or intradural white matter, and configured to acquire or apply an electrical signal on a surface of the spinal cord. The flexible electrode 100 may further include one or more implantation portions 120, which may be arranged symmetrically or asymmetrically and respectively extend from the attachment portion, and may be configured to be capable of being implanted inside the spinal cord or a neural tissue (such as a nerve root, a ganglion, etc.) connected to the spinal cord and be configured to acquire or apply electrical signals at corresponding positions inside the spinal cord. Since the implantation positions and implantation methods of the attachment portion 110 and the implantation portion 120 are different, the strength, adhesion, ductility, etc., required to be achieved by the attachment portion 110 and the implantation portion 120 may be different. Specifically, the attachment portion 110 and the implantation portion 120 may have different thicknesses, or may also be made of different materials. Of course, the attachment portion 110 and the implantation portion 120 may also have the same thickness and be made of the same material. In addition, the flexible electrode may further include a back-end portion 130, which may be used to be engaged with the flexible electrode 100 and a back-end circuit for back-end adaptation, and the attachment portion 110 may extend from the back-end portion 130. The flexible electrode 100 has good flexibility, so it can conform to the position where it is attached or implanted when applied to the spinal cord. The flexible electrode 100 shown in FIG. 1 includes the attachment portion 110 having an elongated shape, and ten implantation portions 120 symmetrically arranged on two long sides of the attachment portion 110. However, it should be understood that FIG. 1 shows only a non-limiting example, and the flexible electrode for the spinal cord may have the attachment portion 110 of different shape and size and the implantation portion 120 of different number, shape, size, and arrangement, as required.

Although the flexible electrode 100 shown in FIG. 1 includes both the attachment portion 110 and the implantation portions 120, it should be understood that the flexible electrode in the present disclosure may also include only the attachment portion configured to be capable of being attached outside or inside dura mater without including the implantation portion configured to be capable of being implanted inside the spinal cord or the neural tissue connected to the spinal cord.

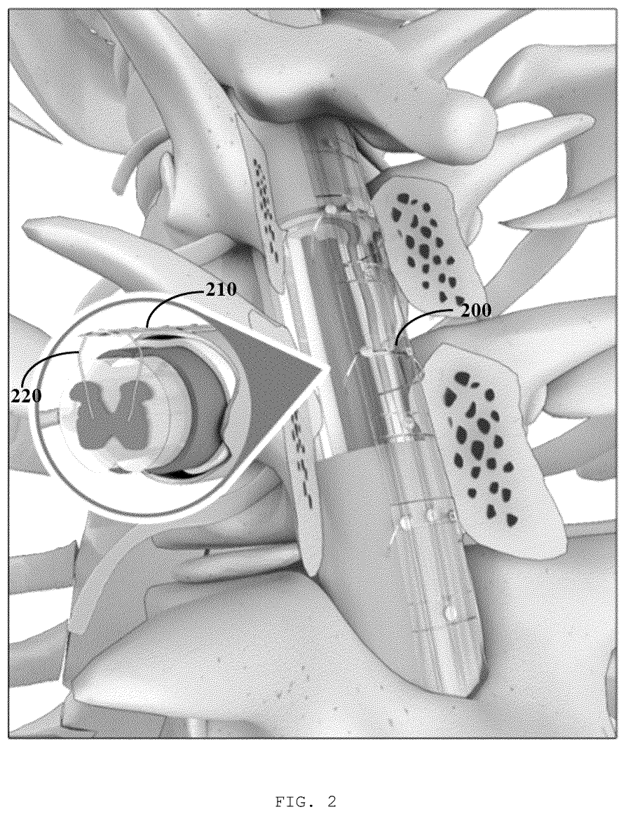

FIG. 2 shows a schematic diagram of an implantation method of at least a part of a flexible electrode 200 for spinal cord according to an embodiment of the present disclosure, and in particular, an enlarged view of the electrode 200 after being implanted. As shown in FIG. 2, when the flexible electrode 200 is implanted into the spine, an attachment portion 210 may be parallel to a nerve bundle in the spine, and an implantation portion 220 may be perpendicular to the nerve bundle or form an angle relative to a vertical direction. The attachment portion 210 and each implantation portion 220 may each include one or more electrode sites, and the electrode sites may be used to acquire electrical signals from spinal nerves or apply electrical signals to the spinal nerves. In this way, when the flexible electrode 200 is used as a recording electrode, the electrode sites of the attachment portion 210 can record local field potential (LFP) signals on the spinal dura mater or an outer part of the white matter that is in contact therewith, and the electrode sites of the implantation portion 220 can record Spike signals of neurons in the spinal nerves, nerve roots and ganglia that are in contact therewith, so as to use the flexible electrode 200 to record the local field potential signals and the Spike signals at the same time; and when the flexible electrode 200 is used as a stimulation electrode, the electrode sites of the attachment portion 210 can apply electrical signals to the spinal dura mater that is in contact therewith, and the electrode sites of the implantation portion 220 can apply electrical signals to the spinal cord that is in contact therewith. FIG. 2 shows an embodiment of implanting the flexible electrode 200 into multiple spinal segments, but it should be understood that the present disclosure is not limited thereto. Instead, the size, shape, arrangement of electrode sites, etc., of the flexible electrode may be adjusted as needed so that the flexible electrode is suitable for one or more or all of the spinal nerves when implanted into one or more or all of the spinal segments, so as to achieve electrophysiological signal recording and electrical stimulation of neurons in one or more or all of the segments of spinal cord.

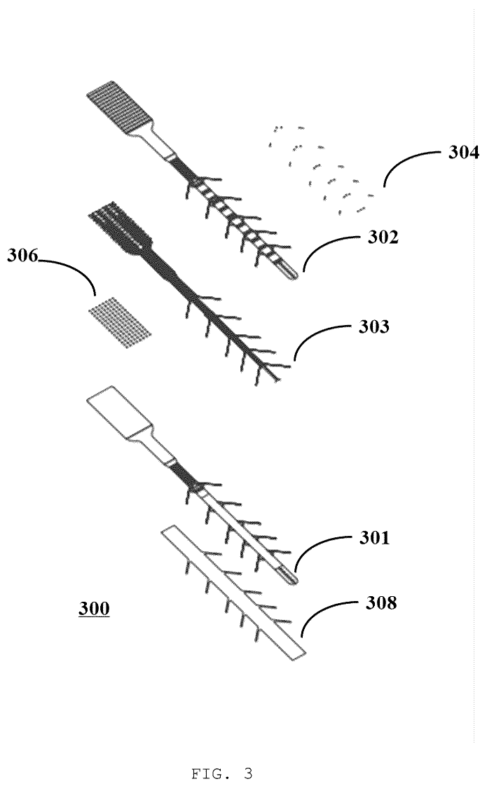

FIG. 3 shows an exploded view of at least a part of a flexible electrode 300 according to an embodiment of the present disclosure. It can be clearly seen from FIG. 3 that the flexible electrode 300 has a multi-layer structure, specifically including a bottom insulating layer 301, a top insulating layer 302, a wire layer 303, an electrode site layer 304, a back-end site layer 306, and a flexible separation layer 308, etc. It should be understood that the layers of the flexible electrode 300 shown in FIG. 3 are only non-limiting examples, and the flexible electrode in the present disclosure may not include one or more of these layers, or may include more other layers. The flexible electrode 300 may include an insulating layer 301 at the bottom and an insulating layer 302 at the top. Specifically, as shown in FIG. 3, the attachment portion, the implantation portion and the back-end portion of the flexible electrode 300 may include the insulating layers 301 and 302. An insulating layer in the flexible electrode may refer to an outer surface layer in the electrode that plays an insulating role. Since the insulating layer of the flexible electrode needs to be in contact with a biological tissue after implantation, a material of the insulating layer is required to have good biocompatibility while having good insulation property. In an embodiment of the present disclosure, the material of the insulating layers 301 and 302 may include polyimide (PI), polydimethylsiloxane (PDMS), Parylene, epoxy resin, polyamide imide (PAI), SU-8 photoresist, silica gel, silicone rubber, etc. In an embodiment according to the present disclosure, in order to make the flexible electrode further have biodegradability, the material of the insulating layers 301 and 302 may also include polylactic acid, polylactic acid—glycolic acid copolymer, etc. In addition, the insulating layers 301 and 302 are also main parts that provide strength in the flexible electrode 300. If the insulating layer is too thin, the strength of the electrode will be reduced; and if the insulating layer is too thick, the flexibility of the electrode will be reduced. In addition, the implantation of an electrode including an overly thick insulating layer will cause greater damage to the organism. In an embodiment according to the present disclosure, the thickness of the insulating layers 301 and 302 may be 100 nm to 300 μm, preferably 300 nm to 20 μm.

The flexible electrode 300 may further include wires in the wire layer 303 between the bottom insulating layer 301 and the top insulating layer 302. In an embodiment according to the present disclosure, the flexible electrode 300 may include one or more wires in the same wire layer 303, wherein each wire may be electrically coupled to an electrode site in the electrode site layer 304 and electrically coupled to a back-end site in the back-end site layer 306. In an embodiment of the present disclosure, the thickness of the wire layer 303 and each wire therein may be 5 nm to 200 μm. The spacing between the wires may be as low as 10 nm, for example. The line width of the wire and the spacing between the wires may be, for example, 10 nm to 500 μm, for example, preferably 100 nm to 30 μm. It should be understood that the size and the like of the wire is not limited to the above-listed ranges, but may vary according to design requirements.

In an embodiment according to the present disclosure, the wire in the wire layer 303 may have a thin film structure including multiple sub-layers stacked in the thickness direction. The materials of these sub-layers may be materials that can enhance the adhesion, ductility, conductivity, etc., of the wires. As a non-limiting example, the wire layer 303 may be a metal film including three sub-layers stacked, wherein a first sub-layer and a second sub-layer that are in contact with the insulating layers 301 and 302 respectively, are adhesion sub-layers, and may adopt metal adhesive materials such as titanium (Ti), titanium nitride (TiN), chromium (Cr), tantalum (Ta) or tantalum nitride (TaN) or non-metal adhesive materials; and a third sub-layer located between the first sub-layer and the second sub-layer is a conductive sub-layer, and may adopt materials with good conductivity such as gold (Au), platinum (Pt), iridium (Ir), tungsten (W), platinum-iridium alloy, titanium alloy, graphite, carbon nanotubes, PEDOT, etc. In an embodiment according to the present disclosure, in order to make the flexible electrode further have biodegradability, the conductive sub-layer may also adopt materials such as magnesium (Mg), molybdenum (Mo) and alloys thereof. It should be understood that the wire layer may be made of other metal or non-metal materials that have conductivity, or may be made of polymeric conductive materials and composite conductive materials. In an embodiment according to the present disclosure, the thickness of the adhesion sub-layer may be 1 nm to 50 nm, and the thickness of the conductive sub-layer may be 5 nm to 200 μm.

The flexible electrode 300 may also include electrode sites in the top electrode site layer 304 located above the top insulating layer 302, and each of the electrode sites is electrically coupled to one of the wires in the wire layer 303 and is in contact with the spinal cord after the flexible electrode 300 is implanted, to acquire an electrical signal from the spinal nerve and transmit the acquired electrical signal via the wire, or to apply an electrical signal received via the wire to the spinal nerve. In the flexible electrode 300 shown in FIG. 3, the attachment portion and each of the implantation portions both include a plurality of corresponding electrode sites. However, it should be understood that the present disclosure is not limited thereto, and each implantation portion of the flexible electrode may include multiple electrode sites for applying or acquiring signals inside the spinal cord as needed, and the attachment portion of the flexible electrode may include multiple electrode sites for applying or acquiring signals on the surface of the spinal cord as needed. In addition, since each electrode site is coupled to its corresponding wire, when the flexible electrode 300 is used as a stimulation electrode, each of the electrode sites can synchronously or asynchronously apply the same or different electrical signals at a deep part and/or at different locations of the surface; and when the flexible electrode 300 is used as a recording electrode, these electrode sites can simultaneously and finely acquire electrical signals at a deep part and/or at different locations of the surface.

In the flexible electrode 300, the electrode site in the top electrode site layer 304 may be electrically coupled to the corresponding wire via a through hole at a position corresponding to the electrode site in the top insulating layer 302. In the case where the flexible electrode includes a plurality of wires, the flexible electrode may correspondingly include a plurality of electrode sites in the top electrode site layer 304, and each of these electrode sites is electrically coupled to one of the plurality of wires via a corresponding through hole in the top insulating layer 302. In an embodiment according to the present disclosure, the electrode sites in the top electrode site layer 304 may have a thin film structure including a plurality of sub-layers stacked in the thickness direction. The material of an adhesion sub-layer close to the wire layer 303 among the plurality of sub-layers may be a material that can enhance the adhesion between the electrode sites and the wires, and the thickness of the adhesion sub-layer may be 1 nm to 50 nm. As a non-limiting example, the electrode site layer 304 may be a metal film including two superimposed sub-layers, wherein a first sub-layer close to the wire layer 303 is Ti, TiN, Cr, Ta, or TaN, and a second sub-layer of the electrode site layer 304 that is exposed to the outside is Au. It should be understood that the electrode site layer may also be similar to the wire layer and may be made of other metal or non-metal materials that have conductivity, such as Pt, Ir, W, Mg, Mo, platinum-iridium alloy, titanium alloy, graphite, carbon nanotubes, PEDOT, etc.

Each electrode site may have a planar size at the micrometer level and a thickness at the nanometer level. In the embodiments according to the present disclosure, the electrode site may be shaped to have various regular or irregular shapes as required, and a number of the electrode sites may be one or more. The electrode site of the attachment portion may have a maximum side length or diameter of 1 μm to 2 mm, and a spacing between the electrode sides may be 10 μm to 20 mm. The electrode site of the implantation portion may be shaped to have various regular or irregular shapes as required, a maximum side length or diameter of the electrode site may be 1 μm to 500 μm, and a spacing between the electrode sites may be 1 μm to 5 mm. It should be understood that the shape, number, size and spacing of the electrode sites may be selected according to the situation of the biological tissue area required to be recorded or stimulated.

In an embodiment according to the present disclosure, the surface of the electrode site that is exposed to the outside and in contact with the biological tissue may also have a surface modification layer to improve the electrochemical properties of the electrode site. As a non-limiting example, the surface modification layer may be obtained by using an electro-induced polymerization coating of PEDOT:PSS, sputtering an iridium oxide film, and the like, for reducing impedance (such as electrochemical impedance at an operating frequency of 1 kHz) in a case where the flexible electrode acquires electrical signals, and for improving charge injection capability in a case where the flexible electrode applies electrical signals for stimulation, thereby improving interaction efficiency.

In an embodiment according to the present disclosure, although not shown in FIG. 3, the flexible electrode may further include an electrode site in a bottom electrode site layer 305 located below the bottom insulating layer 301, and the electrode site may contact a biological tissue after the flexible electrode is implanted to directly acquire or apply an electrical signal. Similar to the electrode sites in the top electrode site layer 304, in the flexible electrode 300, electrode sites in the bottom electrode site layer 305 may be electrically coupled to corresponding wires via through holes in the bottom insulating layer 301 at positions corresponding to the electrode sites. In an embodiment according to the present disclosure, the electrode site in the bottom electrode site layer 305 and the electrode site in the top electrode site layer 304 may be located at opposite positions on the bottom and top sides of the flexible electrode 300, and may be electrically coupled to the same wire in the wire layer 303. In an embodiment according to the present disclosure, the electrode site in the bottom electrode site layer 305 and the electrode site in the top electrode site layer 304 may also be located at different positions on the bottom and top sides of the flexible electrode 300 to acquire or apply an electrical signal in different areas of a biological tissue; and in an embodiment according to the present disclosure, the electrode site in the bottom electrode site layer 305 and the electrode site in the top electrode site layer 304 may also be electrically coupled to different wires in the wire layer 303.

It should be understood that the bottom electrode site layer 305 is an optional part but not a necessary part of the flexible electrode. For example, the flexible electrode in the present disclosure may include only the top electrode site layer 304 without including the bottom electrode site layer 305. The shape, size, material, etc. of the bottom electrode sites may be similar to those of the top electrode sites and will not be described in detail here.

In an embodiment of the present disclosure, the flexible electrode may further include an additional wire layer, that is, the flexible electrode in the present disclosure may include one or more wire layers. The size, material, fabrication method, etc. of the additional wire layer may be similar to those of the wire layer 303, and will not be described in detail here. In the case where the flexible electrode includes an additional wire layer, these wire layers may be separated from each other by an additional insulating layer, and the size, material, and fabrication method of the additional insulating layer may be similar to those of the bottom insulating layer 301 and/or the top insulating layer 302, and will not be described in detail here. One or more wires in these additional wire layers may be electrically coupled to electrode sites located below the bottom insulating layer or above the top insulating layer via through holes in one or more of the bottom insulating layer, the top insulating layer, and the additional insulating layers. By including multiple wire layers in the flexible electrode, the number and accuracy of signals transmitted through the flexible electrode can be increased with the same section width, that is, a high-accuracy and multi-channel electrode is provided, which is conducive to achieving high-throughput interaction.

In an embodiment according to the present disclosure, the back-end portion of the flexible electrode 300 may include a back-end site in the back-end site layer 306, and the back-end site may be electrically coupled to the back-end circuit and one of the wires via a through hole in the bottom insulating layer 301 and/or the top insulating layer 302, so as to achieve bidirectional signal transmission between the electrode site electrically coupled to the wire and the back-end circuit. Here, the back-end circuit may refer to a circuit at the back end of the flexible electrode, such as a signal recording circuit, a signal processing circuit, a signal generating circuit, etc., associated with the signal of the flexible electrode. Preferably, the back-end site layer 306 may be located between the wire layer 303 and at least one of the top insulating layer 302 or the bottom insulating layer 301. In an embodiment according to the present disclosure, the flexible electrode may be coupled to the back-end circuit in a connection manner. Specifically, the ball grid array (BGA) package sites as back-end sites can be adapted to a commercial signal recording system through a printed circuit board (PCB), a flexible printed circuit (FPC), etc. Before the adapted connection, the flexible electrode may be first released from the substrate (for example, to separate the flexible electrode from the substrate by directly peeling the flexible electrode from the substrate, or by removing the flexible separation layer), and the back-end portion is connected to the back-end circuit by a connection method such as solder ball attachment or anisotropic conductive film bonding (ACF Bonding), and then packaging is performed using silica gel or the like. In an embodiment according to the present disclosure, the flexible electrode may also be integrated with the back-end circuit, that is, the back-end portion of the flexible electrode is first connected to the back-end circuit, and then they are separated from the substrate as a whole. Specifically, pre-processing functions such as signal amplification and filtering may be integrated on a dedicated chip, and then the chip is connected and packaged with an integrated PCB at the back end of the flexible electrode by bonding or the like, so as to achieve wireless transmission, and charging, etc. In this case, an independent flexible electrode and an independent dedicated chip as the back-end circuit may be used, and the electrical connection between the flexible electrode and the dedicated chip may be performed through solder ball attachment, or ACF Bonding, etc. Alternatively, a certain space may be reserved on a wafer of a chip as the back-end circuit, for which tape-out has been done in advance, and the electrode may be directly manufactured on this basis, thereby realizing the joint processing or separate processing of the chip and the electrode to achieve a higher level of integration.

The back-end site may have a planar size at the micrometer level and a thickness at the nanometer level. As a non-limiting example, the back-end site may be the BGA package site with a diameter of 50 μm to 2000 μm, or may be a circular, elliptical, rectangular, rounded rectangular, or chamfered rectangular site with a side length of 50 μm to 2000 μm, and the thickness of the back-end site layer 306 and the back-end sites therein may be 5 nm to 200 μm. It should be understood that the shape, size, etc. of the back-end sites are not limited to the ranges listed above, but may vary according to design requirements.

The back-end site for connection may include multiple sub-layers in the thickness direction, a material of an adhesion sub-layer close to the wire layer 303 among the multiple sub-layers may be a material that can enhance the adhesion between the electrode site and the wire; a material of a flux sub-layer in the middle among the multiple sub-layers may be a flux material; a material of a conductive sub-layer among the multiple sub-layers may be other conductive metal or non-metallic material such as that of the wire layer mentioned above; and an outermost layer among the multiple sub-layers that may be exposed through the insulating layers 301 and 302 is an anti-oxidation protective sub-layer. As a non-limiting example, the back-end site layer 306 may be a metal film including three superimposed sub-layers, wherein a first sub-layer close to the wire layer 303 may be an adhesion sub-layer at the nanometer-scale to improve the adhesion between the back-end site layer 306 and the wire layer 303. The material of the first sub-layer as the adhesion sub-layer may be any one of chromium, tantalum, tantalum nitride, titanium or titanium nitride or a combination thereof, the material of a second sub-layer as the flux sub-layer may be nickel (Ni), Pt or palladium (Pd), and the material of a third sub-layer as the conductive sub-layer may be Au, Pt, Ir, W, Mg, Mo, platinum-iridium alloy, titanium alloy, graphite, carbon nanotubes, PEDOT, etc. It should be understood that the back-end site layer may also be made of other metal or non-metallic materials that have conductivity. The back-end site layer 306 in FIG. 3 serves as a part connected to a back-end processing system or chip, and the design of the size, spacing, shape, etc., of sites therein may be changed according to different connection manners at the back end.

In an embodiment according to the present disclosure, the flexible electrode may not include the site layers such as the top electrode site layer, the bottom electrode site layer, or the back-end site layer, etc. In this case, the electrode sites on the electrode and the back-end sites in the back-end portion for adaptation may both be parts of the wire layer, and electrically coupled to corresponding wires in the wire layer. That is, the flexible electrode may not include the top electrode site layer, the bottom electrode site layer, or the back-end site layer, so the electrode sites and the back-end sites are both implemented in the wire layer. Furthermore, the electrode sites for sensing and applying electrical signals may directly contact the tissue area into which the electrode is implanted. As a non-limiting example, each electrode site may be electrically coupled in the wire layer to a corresponding wire in the wire layer, and exposed at the outer surface of the electrode via a corresponding through hole in the top insulating layer or the bottom insulating layer and in contact with a biological tissue. Similarly, in an embodiment according to the present disclosure, the back-end site may also be adaptedly connected to the back-end circuit via a corresponding through hole in the top insulating layer or the bottom insulating layer, and in this case the flexible electrode may not include a separate back-end site layer.

In an embodiment according to the present disclosure, the flexible electrode 300 may further include the flexible separation layer 308. The flexible separation layer 308 of the flexible electrode 300 in FIG. 3 is shown as the lowest layer of the entire flexible electrode, but it should be understood that the position of the flexible separation layer is not limited thereto, and the flexible electrode may include therein one or more flexible separation layers located at different positions. Preferably, the flexible separation layer may be formed between the substrate and the bottom insulating layer. The flexible separation layer may be made of a material that can be removed by a specific substance (such as a solution) to separate two parts of the flexible electrode above and below the flexible separation layer while avoiding damage to the flexible electrode. In an embodiment of the present disclosure, the material of the flexible separation layer may be a metal such as Ni, Cr, or aluminum (Al), etc., or non-metal material. It should be understood that the flexible separation layer is an optional part but not a necessary part of the flexible electrode, and in a case where the flexible separation layer is absent, the flexible electrode may be manufactured to be directly separable from the substrate. In an embodiment according to the present disclosure, the flexible separation layer 308 further includes an adhesion sub-layer, and the material of the adhesion sub-layer may be chromium, tantalum, tantalum nitride, titanium or titanium nitride.

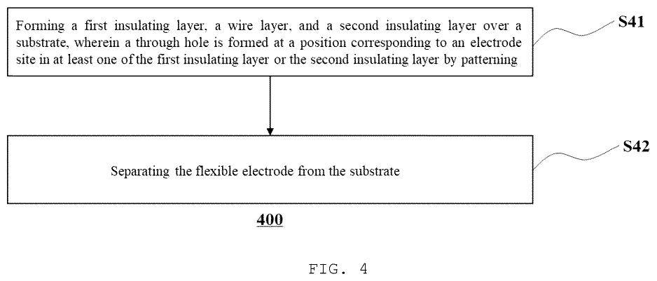

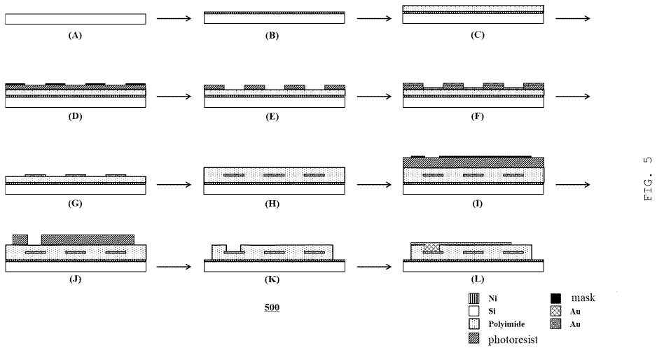

FIG. 4 shows a flowchart of a method 400 for manufacturing a flexible electrode according to an embodiment of the present disclosure. In the present disclosure, a manufacturing method based on a micro-electro mechanical system (MEMS) process may be adopted to manufacture a nano-scale flexible electrode. As shown in FIG. 4, the method 400 may include: at S41, forming a first insulating layer, a wire layer, and a second insulating layer over a substrate, wherein a through hole is formed at a position corresponding to an electrode site in at least one of the first insulating layer or the second insulating layer by patterning; and at S42, separating the flexible electrode from the substrate. The steps of forming the layers of the flexible electrode at S41 are described in detail below in conjunction with FIGS. 5 to 7.

FIG. 5 shows a schematic diagram of a method 500 for manufacturing a flexible electrode according to an embodiment of the present disclosure, wherein the attachment portion and the implantation portion of the flexible electrode have the same thickness and are made of the same material, and the flexible electrode includes at least the flexible separation layer, the bottom insulating layer, the wire layer, the top insulating layer, and the top electrode site layer. The forming process and structures of the flexible separation layer, bottom insulating layer, wire layer, top insulating layer, electrode site layer, etc., of the flexible electrode are described in more detail in conjunction with FIG. 5.

The view (A) of FIG. 5 shows a substrate of the electrode. In an embodiment according to the present disclosure, a hard substrate such as glass, quartz, silicon wafer, etc., may be used. In an embodiment according to the present disclosure, other soft materials may also be used as the substrate, such as the same material as that of the insulating layer.

The view (B) of FIG. 5 shows a step of forming a flexible separation layer on the substrate. The flexible separation layer may be removed by applying a specific substance, so as to facilitate the separation of the flexible portion of the electrode from the hard substrate. In the embodiment shown in FIG. 5, Ni is used as the material of the flexible separation layer, and other materials such as Cr and Al may also be used. In an embodiment according to the present disclosure, when a flexible separation layer is formed on the substrate by evaporation, a portion of the exposed substrate may be etched first to improve the flatness of the entire substrate after evaporation. It should be understood that the flexible separation layer is an optional part but not a necessary part of the flexible electrode. Depending on the characteristics of the selected material, the flexible electrode may also be conveniently separated without the flexible separation layer. In an embodiment according to the present disclosure, the flexible separation layer may also be provided with a mark which may be used for alignment of subsequent layers.

The view (C) of FIG. 5 shows forming a bottom insulating layer on the flexible separation layer. As a non-limiting example, in the case where the insulating layer is made of the polyimide material, the forming of the bottom insulating layer may include steps such as a film forming process, a formed-film curing step, and an enhanced curing to form a thin film as the insulating layer. The film forming process may include applying polyimide on the flexible separation layer, for example, a layer of polyimide may be formed by spin coating in segmented rotational speeds. The formed-film curing may include gradually heating to a high temperature and keeping the temperature to form a film, so as to perform subsequent processing steps. The enhanced curing may include multi-gradient temperature increasing before forming subsequent layers, preferably in a vacuum or nitrogen atmosphere, and baking for several hours. It should be understood that the above forming process is only a non-limiting example of the forming process of the bottom insulating layer, and one or more of the steps may be omitted, or more other steps may be included.

It should be noted that the above forming process is directed to an embodiment where a bottom insulating layer is formed in a flexible electrode without a bottom electrode site layer and there is no through hole corresponding to the electrode sites in the bottom insulating layer. If the flexible electrode includes a bottom electrode site layer, the bottom electrode site layer may be formed on the flexible separation layer before forming the bottom insulating layer. For example, Au and Ti may be sequentially evaporated on the flexible separation layer. The step of patterning the bottom electrode sites will be described in detail later with respect to the top electrode sites. Accordingly, in the case where the flexible electrode includes bottom electrode sites, in the process of forming the bottom insulating layer, in addition to the above steps, a patterning step may also be included for etching through holes at positions corresponding to the bottom electrode sites in the bottom insulating layer. The step of patterning the insulating layer will be described in detail later with respect to the top insulating layer.

Views (D) to (G) of FIG. 5 show forming a wire layer on the bottom insulating layer. As shown in the view (D), a photoresist and a mask may be applied on the bottom insulating layer. It should be understood that other photolithography methods may also be used to prepare the patterned thin film, such as laser direct writing and electron beam photolithography. In an embodiment according to the present disclosure, for a metal film such as the wire layer, a double-layer photoresist may be applied to facilitate the forming (evaporation or sputtering) and stripping of the patterned thin film. By setting the pattern of the mask associated with the wire layer, for example, the pattern of the wire layer 303 shown in FIG. 3 may be implemented, that is, the contour of one or more wires in the electrode extending from the back-end portion. Then, exposure and development may be performed to obtain a structure as shown in the view (E). In an embodiment according to the present disclosure, the exposure may be carried out by contact photolithography, and the mask and the structure are exposed in a vacuum contact mode. In an embodiment according to the present disclosure, different developers and concentrations thereof may be adopted for patterns of different sizes. This step may also include alignment between the layers. Next, a film may be formed on the structure shown in the view (E), for example, processes such as evaporation and sputtering may be used to deposit a metal thin film material, such as Au, to obtain a structure shown in the view (F). Next, stripping may be performed to separate the thin film in the non-patterned area from the thin film in the patterned area by removing the photoresist in the non-patterned area, so as to obtain a structure as shown in the view (G), that is, to form a wire layer. In an embodiment according to the present disclosure, a photoresist removal may be performed again after the photoresist stripping, so as to further remove the residual photoresist on the surface of the structure.

In an embodiment according to the present disclosure, a back-end site layer may also be formed before forming the wire layer. As a non-limiting example, the forming process of the back-end site layer may be similar to that of the metal film described above with respect to the wire layer.

Views (H) to (K) of FIG. 5 show forming a top insulating layer. For photosensitive thin films, patterning may be generally achieved directly through patterned exposure and development, while for non-photosensitive materials used in the insulating layer, patterning cannot be achieved by exposing and developing the materials themselves. In this case, a sufficiently thick patterned anti-etching layer may be formed on this layer, and then the thin film in an area not covered by the anti-etching layer is removed by dry etching (the anti-etching layer will also be thinned at the same time, so it is necessary to ensure that the anti-etching layer is thick enough), and then the anti-etching layer is removed to achieve the patterning of the non-photosensitive layer. As a non-limiting example, when forming the insulating layer, the photoresist may be used as the anti-etching layer. The forming of the top insulating layer may include steps such as a film forming process, formed-film curing, patterning, and enhanced curing, wherein the view (H) shows a structure obtained after the film-forming of the top insulating layer, the view (I) shows the application of photoresist and mask on the top insulating layer after the film-forming, the view (J) shows a structure including the anti-etching layer obtained after exposure and development, and the view (K) shows a structure including the top insulating layer formed. The film forming process, formed-film curing and enhanced curing have been described in detail above with respect to the bottom insulating layer, and description therefor is omitted here for brevity. The patterning step may be performed after the formed-film curing, or after the enhanced curing. After the enhanced curing, the insulating layer has a stronger anti-etching ability. Specifically, in the view (I), a sufficiently thick layer of photoresist is applied on the insulating layer through steps such as spin coating and baking. By providing the pattern of the mask related to the top insulating layer, for example, the pattern of the top insulating layer 302 shown in FIG. 3 may be realized, that is, the contour of the top insulating layer implemented on the one or more wires in the electrode extending from the back-end portion and the contours of the through holes implemented at positions corresponding to the electrode sites in the top insulating layer. In the view (J), the pattern is transferred to the photoresist on the insulating layer through steps such as exposure and development to obtain an anti-etching layer, wherein the portion to be removed from the top insulating layer is exposed. The exposed portion of the top insulating layer may be removed by oxygen plasma etching, to obtain a structure shown in the view (K).

In the embodiment according to the present disclosure, an adhesion enhancement treatment may be performed before forming the top insulating layer, so as to improve the bonding force between the bottom insulating layer and the top insulating layer.

The view (L) of FIG. 5 shows forming a top electrode site layer above the top insulating layer by evaporation or the like.

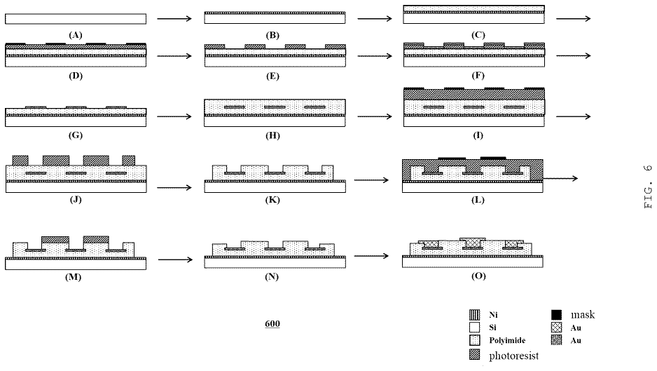

FIG. 6 shows a schematic diagram of a method 600 for manufacturing a flexible electrode according to an embodiment of the present disclosure, in which the attachment portion and the implantation portion have different thicknesses, and the flexible electrode includes at least the flexible separation layer, the bottom insulating layer, the wire layer, the top insulating layer and the top electrode site layer.

Views (A) to (G) of FIG. 6 showing the forming of the flexible separation layer, the bottom insulating layer, and the wire layer are similar to views (A) to (G) of FIG. 5, and are not described again herein.

Views (H) to (K) of FIG. 6 show forming the top insulating layer. For the sake of brevity, those in the forming process of the top insulating layer similar to those of views (H) to (K) of FIG. 5 are not repeated here. Here, in order to make the attachment portion and the implantation portion have different thicknesses, the top insulating layers of the attachment portion and the implantation portion are formed to have different thicknesses. The view (H) of FIG. 6 shows a structure obtained after the film-forming of the top insulating layer. The view (I) of FIG. 6 shows the application of photoresist and mask on the top insulating layer after the film-forming, wherein the pattern of the mask is set to be related to the top insulating layer, for example, the pattern of the top insulating layer 302 shown in FIG. 3 may be implemented, that is, the contour of the top insulating layer implemented on one or more wires in the electrode extending from the back-end portion and the contours of the through holes implemented at the positions corresponding to the electrode sites in the top insulating layer. The view (J) of FIG. 6 shows a structure including an anti-etching layer obtained after exposure and development. The view (K) of FIG. 6 shows a structure of the top insulating layer after etching, at this time, the top insulating layers of the attachment portion and the implantation portion have the same thickness. The view (L) of FIG. 6 shows that a photoresist and a mask are applied again on the etched top insulating layer of the view (K), wherein the pattern of the mask is set to be related to the top insulating layer of the attachment portion, for example, the pattern of the top insulating layer 302 of the attachment portion shown in FIG. 3 may be implemented. The view (M) of FIG. 6 shows a structure including an anti-etching layer obtained after exposing and developing again, which is located on the insulating layer of the attachment portion to protect the insulating layer of the attachment portion and expose the insulating layer of the implantation portion. The view (N) of FIG. 6 shows the final top insulating layer obtained after etching, wherein a portion of the insulating layer of the implantation portion is etched away, so that its thickness is less than that of the insulating layer of the attachment portion.

The view (O) of FIG. 6 shows a top electrode site layer formed on the top insulating layer by evaporation or the like.

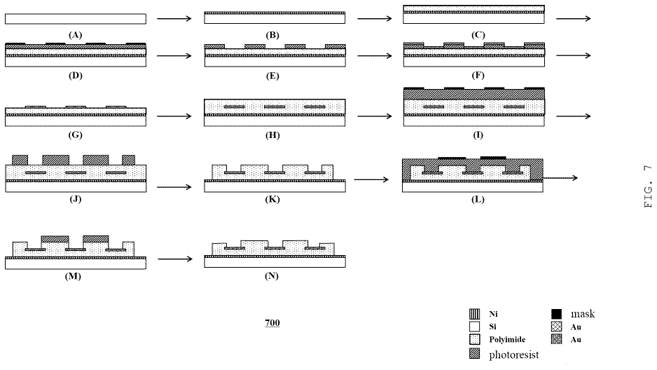

FIG. 7 shows a schematic diagram of a method 700 for manufacturing a flexible electrode according to an embodiment of the present disclosure, in which the attachment portion and the implantation portion have different thicknesses, and the flexible electrode includes at least the flexible separation layer, the bottom insulating layer, the wire layer and the top insulating layer, but does not include the electrode site layer.

Views (A) to (N) of FIG. 7 are similar to views (A) to (N) of FIG. 6, but it should be noted that, unlike FIG. 5 and FIG. 6, the mask in the view (D) has a pattern related to wires, electrode sites, and back-end sites, so that the wire layer of the manufactured flexible electrode includes the wires of the flexible electrode, electrode sites for acquiring or applying electrical signals, and back-end sites for adaptedly connecting to the back-end circuit. In addition, unlike FIG. 5 and FIG. 6, the method shown in FIG. 7 does not include the step of forming the top electrode site layer.

The present disclosure provides a flexible electrode for spinal cord and a method for manufacturing the same. The flexible electrode has both deep and surface structures and can be attached outside the dura mater or implanted into the spinal cord; and it can be used for acquiring electrical signals from the spinal cord while recording epidural and spinal LFP and Spike signals, and also can be used for intraspinal and epidural functional electrical stimulation to the spinal cord. By proportionally increasing or decreasing the size of the electrode, it can be adapted to different human bodies or other vertebrates; and electrodes with different numbers of layers, sizes, shapes, numbers of electrode sites, and arrangements of electrode site can be designed according to needs and manufactured using nanomanufacturing technology. The electrode has good spatial resolution and high channel count, and can record a large amount of neuronal activity for further motor decoding and fine limb manipulation. The material adopted for this flexible electrode can significantly reduce the stiffness of the electrode, thereby effectively avoiding breakage and providing a long-term stable spinal nerve interface when applied to spinal cord that has been repeatedly deformed. The material adopted for this flexible electrode only causes a slight immune response after implantation, and the flexible electrode has an ultra-thin structure that can avoid microenvironment deterioration and peripheral neuron necrosis, thereby further improving its biocompatibility and chronic stability.

This flexible electrode has good application prospects and value in neuroscience research and rehabilitation medicine applications.

The words “front”, “rear”, “top”, “bottom”, “above”, “below”, etc., if present, in the specification and claims are used for descriptive purposes and are not necessarily used to describe an invariant relative position. It should be understood that the words so used are interchangeable where appropriate, such that the embodiments of the present disclosure described herein, for example, are capable of operation in other orientations than those illustrated or otherwise described herein.

As used herein, the word “exemplary” means “used as an example, instance, or illustration” rather than serving as a “model” to be exactly copied. Any implementation described as an example herein is not necessarily to be construed as preferred or advantageous over other implementations. Moreover, the present disclosure is not limited by any stated or implied theory given in the above sections of technical field, background, summary, or detailed description.

As used herein, the term “substantially” is intended to include any minor variations due to design or manufacturing imperfections, device or component tolerances, environmental influences, and/or other factors. The term “substantially” also allows for deviations from a perfect or ideal situation due to parasitic effects, noise, and other practical considerations that may exist in actual implementations.

The terms “first”, “second” and the like may be used herein for reference purposes only and are not intended to be limiting. For example, the terms “first”, “second” and other such numerical terms referring to structures or elements do not imply a sequence or order unless the context clearly indicates otherwise.

It should also be understood that when the term “include/comprise” is used herein, it indicates the presence of the stated features, entities, steps, operations, units and/or components, but does not exclude the presence or addition of one or more other features, entities, steps, operations, units and/or components and/or combinations thereof.

As used herein, the term “and/or” includes any and every combinations of one or more of the associated listed items. The terms used herein are for the purpose of describing specific embodiments only and are not intended to limit the present disclosure. As used herein, the singular forms “a”, “an”, and “the” are also intended to include the plural forms, unless the context clearly indicates otherwise.

Those skilled in the art will appreciate that the boundaries between the above operations are merely illustrative. Multiple operations may be combined into a single operation, a single operation may be distributed in additional operations, and operations may be performed at least partially overlapping in time. Moreover, alternative embodiments may include multiple instances of a particular operation, and the operation order may be changed in other various embodiments. However, other modifications, variations, and replacements are also possible. Therefore, this specification and accompanying drawings should be considered illustrative, not restrictive.

Although some specific embodiments of the present disclosure have been described in detail by way of example, it should be understood by those skilled in the art that the above examples are for illustration only and are not intended to limit the scope of the present disclosure. The various embodiments disclosed herein may be combined in any manner without departing from the spirit and scope of the present disclosure. It should also be understood by those skilled in the art that various modifications may be made to the embodiments without departing from the scope and spirit of the present disclosure. The scope of the present disclosure is defined by the appended claims.

What is claimed is:

1. A flexible electrode for spinal cord, the flexible electrode comprising an attachment portion configured to be capable of being attached outside an epidural or intradural white matter and configured to acquire or apply an electrical signal on a surface of the spinal cord;
wherein the attachment portion of the flexible electrode comprises a first insulating layer, a second insulating layer, and a wire layer located between the first insulating layer and the second insulating layer; and
wherein the attachment portion of the flexible electrode further comprises one or more electrode sites, each of which is electrically coupled to one of wires in the wire layer, and is in contact with the spinal cord after implantation of the flexible electrode to acquire an electrical signal from a spinal nerve and transmit the acquired electrical signal via the wire, or to apply an electrical signal received via the wire to the spinal nerve.

2. The flexible electrode according to claim 1, wherein the flexible electrode further comprises one or more implantation portions each extending from the attachment portion, the one or more implantation portions being configured to be capable of being implanted inside the spinal cord or inside a neural tissue connected to the spinal cord, and configured to acquire or apply an electrical signal at a corresponding position inside the spinal cord;
wherein the implantation portion comprises a first insulating layer, a second insulating layer and a wire layer located between the first insulating layer and the second insulating layer;
wherein the implantation portion further comprises one or more electrode sites, each of which is electrically coupled to one of wires in the wire layer and is in contact with the spinal cord after the implantation of the flexible electrode to acquire an electrical signal from the spinal nerve and transmit the acquired electrical signal via the wire, or apply an electrical signal received via the wire to the spinal nerve; and
wherein the attachment portion and the implantation portion of the flexible electrode have the same or different thicknesses and are made of the same or different materials.

3. The flexible electrode according to claim 1 or 2, wherein:
the flexible electrode comprises a plurality of wire layers which are separated by an additional insulating layer therebetween, and each of the wire layers comprises therein a plurality of wires spaced apart from each other.

4. The flexible electrode according to claim 1 or 2, wherein:
the electrode site is located in an electrode site layer outside at least one of the first insulating layer or the second insulating layer, and is electrically coupled to the wire in the wire layer via a through hole in the at least one of the first insulating layer or the second insulating layer.

5. The flexible electrode according to claim 4, wherein the electrode site comprises a conductive sub-layer, and a material of the conductive sub-layer is any one of gold, platinum, iridium, tungsten, magnesium, molybdenum, platinum-iridium alloy, titanium alloy, graphite, carbon nanotubes, PEDOT or a combination thereof.

6. The flexible electrode according to claim 5, wherein the electrode site further comprises an adhesion sub-layer close to the wire layer, the adhesion sub-layer being made of a material capable of enhancing adhesion between the electrode site and the wire layer.

7. The flexible electrode according to claim 3, wherein the electrode site is located in the wire layer and is exposed via a through hole in at least one of the first insulating layer or the second insulating layer.

8. The flexible electrode according to claim 2, wherein the electrode site is shaped as required, a number of the electrode sites is one or more, wherein:
the electrode site of the attachment portion has a maximum side length or diameter of 1 micrometer to 2 millimeters, and a spacing between the electrode sites of 10 micrometers to 20 millimeters;
the electrode site of the implantation portion has a maximum side length or diameter of 1 micrometer to 500 micrometers, and a spacing between the electrode sites of 1 micrometer to 5 millimeters.

9. The flexible electrode according to claim 1 or 2, further comprising a back-end portion, wherein:
the attachment portion extends from the back-end portion, and
the back-end portion comprises a back-end site coupled to a back-end circuit and one of the wires in the wire layer to achieve bidirectional signal transmission between the back-end circuit and the electrode site electrically coupled to the one of the wires.

10. The flexible electrode according to claim 9, wherein the flexible electrode is separated from a substrate after the back-end portion is coupled to the back-end circuit, or the back-end portion is coupled to the back-end circuit after the flexible electrode is separated from the substrate.

11. The flexible electrode according to claim 9, wherein:
the back-end site is located in the wire layer and is adaptedly connected to the back-end circuit via a through hole in at least one layer of the first insulating layer or the second insulating layer; or
the back-end site is located in a back-end site layer between the wire layer and at least one of the first insulating layer or the second insulating layer and is electrically coupled to a wire in the wire layer via a through hole in the at least one of the first insulating layer or the second insulating layer.

12. The flexible electrode according to claim 9, wherein the back-end site comprises a conductive sub-layer, and a material of the conductive sub-layer is any one of gold, platinum, iridium, tungsten, magnesium, molybdenum, platinum-iridium alloy, titanium alloy, graphite, carbon nanotubes, PEDOT or a combination thereof.

13. The flexible electrode according to claim 9, wherein the back-end site has a thickness of 5 nanometers to 200 micrometers.

14. The flexible electrode according to claim 12, wherein the back-end site further comprises an adhesion sub-layer close to the wire layer, and a material of the adhesion sub-layer is any one of chromium, tantalum, tantalum nitride, titanium, or titanium nitride or a combination thereof.

15. The flexible electrode according to claim 1 or 2, wherein the wire layer comprises a conductive sub-layer, and a material of the conductive sub-layer is any one of gold, platinum, iridium, tungsten, platinum-iridium alloy, titanium alloy, graphite, carbon nanotubes, PEDOT or a combination thereof.

16. The flexible electrode according to claim 15, wherein the conductive sub-layer has a thickness of 5 nanometers to 200 micrometers.

17. The flexible electrode according to claim 9, wherein the wire layer comprises a conductive sub-layer and an adhesion sub-layer close to any one of the electrode site or the back-end site, and a material of the adhesion sub-layer is any one of chromium, tantalum, tantalum nitride, titanium, or titanium nitride or a combination thereof.

18. The flexible electrode according to claim 1 or 2, wherein the first insulating layer and the second insulating layer have a thickness of 100 nanometers to 300 micrometers.

19. The flexible electrode according to claim 1 or 2, wherein a material of the first insulating layer and the second insulating layer is any one of polyimide, polydimethylsiloxane, parylene, epoxy resin, polyamide imide, SU-8 photoresist, silica gel, silicone rubber, or a combination thereof.

20. The flexible electrode according to claim 1 or 2, further comprising a flexible separation layer, wherein the flexible separation layer is capable of being removed by a specific substance to separate a part of the flexible electrode without damage to the flexible electrode.

21. The flexible electrode according to claim 20, wherein a material of the flexible separation layer is any one of nickel, chromium, aluminum, or a combination thereof.

22. The flexible electrode according to claim 20, wherein the flexible separation layer further comprises an adhesion sub-layer, and a material of the adhesion sub-layer is chromium, tantalum, tantalum nitride, titanium, or titanium nitride.

23. The flexible electrode according to claim 2, wherein the attachment portion is parallel to a nerve bundle in a spine, and the implantation portion is perpendicular to the nerve bundle or forms an angle with respect to a vertical direction.

24. The flexible electrode according to claim 2, wherein:
the electrode site of the attachment portion is configured to be capable of recording a local field potential signal outside the epidural or intradural white matter, and the electrode site of the implantation portion is configured to be capable of recording Spike signals of neurons inside a nerve root, a ganglion and the spinal cord, so as to simultaneously record the local field potential signal and the Spike signals using the flexible electrode; and
the electrode site of the attachment portion is configured to be capable of applying an electrical signal outside the epidural or intradural white matter, and the electrode site of the implantation portion is configured to be capable of partially applying an electrical signal inside the nerve root, the ganglion, and the spinal cord.

25. The flexible electrode according to claim 2, wherein the flexible electrode comprises the attachment portion that is elongated and a plurality of the implantation portions extending from two opposite long sides of the attachment portion.

26. The flexible electrode according to claim 25, wherein the plurality of the implantation portions are arranged symmetrically or asymmetrically on the two long sides.

27. The flexible electrode according to claim 1 or 2, wherein a material of the wire layer is any one of magnesium, molybdenum or alloys thereof or a combination thereof, and a material of the first insulating layer and the second insulating layer is any one of polylactic acid or polylactic acid-glycolic acid copolymer or a combination thereof, so that the flexible electrode is biodegradable.

28. The flexible electrode according to claim 1 or 2, wherein the flexible electrode is configured to be usable in one or more or all of spinal segments, so as to acquire or apply an electrical signal from or to neurons in the one or more or all of the spinal segments, respectively.

29. A manufacturing method for a flexible electrode for spinal cord according to any one of claims 1 to 28, comprising:
forming the first insulating layer, the wire layer, the second insulating layer, and the electrode site over a substrate; and
separating the flexible electrode from the substrate,
wherein a through hole is formed at a position corresponding to the electrode site in at least one of the first insulating layer or the second insulating layer by patterning.

30. The manufacturing method according to claim 29, wherein:
the electrode site is formed to be located in the wire layer and exposed via a through hole in at least one of the first insulating layer or the second insulating layer; or
the electrode site is formed in an electrode site layer located outside at least one of the first insulating layer or the second insulating layer, and is electrically coupled to the wire in the wire layer via a through hole in the at least one of the first insulating layer or the second insulating layer.

31. The manufacturing method according to claim 29, wherein:
a back-end site is formed to be located in the wire layer and adaptedly connected to a back-end circuit via a through hole in at least one of the first insulating layer or the second insulating layer; or
a back-end site is formed to be located in a back-end site layer between the wire layer and at least one of the first insulating layer or the second insulating layer, and is electrically coupled to a wire in the wire layer via a through hole in the at least one of the first insulating layer or the second insulating layer.
